(19) United States
(12) Patent Application Publication
(10) Pub. No.: US 2006/0259088 A1
(43) Pub. Date: Nov. 16, 2006

(54) Method and apparatus for delivering pacing pulses using a coronary stent

(76) Inventors: Joseph M. Pastore, Woodbury, MN (US); Andrew P. Kramer, Stillwater, MN (US); Julio C. Spinelli, Shoreview, MN (US); Rodney W. Salo, Fridley, MN (US); Tamara Colette Baynham, Blaine, MN (US); Jeffrey Ross, Roseville, MN (US); Frits W. Prinzen, Maastricht (NL); Ward Y. R. Vanagt, Maastricht (NL); Richard N. Cornelussen, Maastricht (NL)

Correspondence Address: SCHWEGMAN, LUNDBERG, WOESSNER & KLUTH, P.A., P.O. BOX 2938, MINNEAPOLIS, MN, 55402, US

(21) Appl. No.: 11/129,058

(22) Filed: May 13, 2005

(51) Int. Cl. A61N 1/362

(52) U.S. Cl. 607/9

(57) ABSTRACT

An implantable cardiac protection pacing system delivers pacing pulses to protect the heart from injuries associated with ischemia and myocardial infarction. The system includes an implantable pulse generator (PG) that delivers the pacing pulses and a coronary stent electrically connected to the implantable PG to function as a pacing electrode through which the pacing pulses are delivered. In one embodiment, an intravascular lead provides the electrical connection between the coronary stent and the implantable PG to allow the implantable PG to be implanted in the femoral region. In another embodiment, the coronary stent and the implantable PG are integrated into an intravascular pulse generator-stent.

### CROSS-REFERENCE TO RELATED APPLICATIONS

This application is related to co-pending, commonly assigned, U.S. patent application Ser. No. 10/079,056, entitled “CHRONICALLY-IMPLANTED DEVICE FOR SENSING AND THERAPY,” filed on Feb. 19, 2002, U.S. patent application Ser. No. 11/030,575, entitled “INTERMITTENT AUGMENTATION PACING FOR CARDIOPROTECTIVE EFFECT,” filed on Jan. 6, 2005, U.S. patent application Ser. No. 11/113,828, entitled “METHOD AND APPARATUS FOR PACING DURING REVASCULARIZATION,” filed on Apr. 25, 2005, and U.S. patent application Ser. No. , entitled “METHOD AND APPARATUS FOR CARDIAC PROTECTION PACING,” filed on even date herewith (Attorney Docket No. 279.956US1), which are hereby incorporated by reference in their entirety.

### TECHNICAL FIELD

This document relates generally to cardiac pacing systems and particularly to a system for delivering pacing pulses through an intravascular device such as a coronary stent.

### BACKGROUND

The heart is the center of a person's circulatory system. It includes an electro-mechanical system performing two major pumping functions. The left portions of the heart draw oxygenated blood from the lungs and pump it to the organs of the body to provide the organs with their metabolic needs for oxygen. The right portions of the heart draw deoxygenated blood from the body organs and pump it to the lungs where the blood gets oxygenated. These pumping functions are resulted from contractions of the myocardium. In a normal heart, the sinoatrial node, the heart's natural pacemaker, generates electrical impulses that propagate through an electrical conduction system to various regions of the heart to excite the myocardial tissues of these regions. Coordinated delays in the propagations of the electrical impulses in a normal electrical conduction system cause the various portions of the heart to contract in synchrony to result in efficient pumping functions. A blocked or otherwise abnormal electrical conduction and/or deteriorated myocardial tissue cause dysynchronous contraction of the heart, resulting in poor hemodynamic performance, including a diminished blood supply to the heart and the rest of the body. The condition where the heart fails to pump enough blood to meet the body's metabolic needs is known as heart failure.

Myocardial infarction (MI) is the necrosis of portions of the myocardial tissue resulted from cardiac ischemia, a condition in which the myocardium is deprived of adequate oxygen and metabolite removal due to an interruption in blood supply caused by an occlusion of a blood vessel such as a coronary artery. The necrotic tissue, known as infarcted tissue, loses the contractile properties of the normal, healthy myocardial tissue. Consequently, the overall contractility of the myocardium is weakened, resulting in an impaired hemodynamic performance. Following an MI, cardiac remodeling starts with expansion of the region of infarcted tissue and progresses to a chronic, global expansion in the size and change in the shape of the entire left ventricle. The consequences include a further impaired hemodynamic performance and a significantly increased risk of developing heart failure.

Therefore, there is a need to protect the myocardium from injuries associated with ischemic events, including MI.

### SUMMARY

An implantable cardiac protection pacing system delivers pacing pulses to protect the heart from injuries associated with ischemic events, including MI. The system includes an implantable pulse generator (PG) that delivers the pacing pulses and a coronary stent electrically connected to the implantable PG to function as a pacing electrode through which the pacing pulses are delivered.

In one embodiment, a cardiac pacing system includes an implantable pulse generator and a coronary stent. The implantable pulse generator includes a control circuit and a pulse output circuit. The control circuit includes a cardiac protection pacing timer that times one or more cardiac protection pacing sequences. The one or more cardiac protection pacing sequences each include alternating pacing and non-pacing periods. The pacing periods each have a pacing duration during which a plurality of pacing pulses is delivered. The non-pacing periods each have a non-pacing duration during which no pacing pulse is delivered. The pulse output circuit delivers the plurality of pacing pulses during each of the pacing periods. The coronary stent includes at least one electrode portion electrically connected to the pulse output circuit for delivering the pacing pulses.

In one embodiment, a method for operating a pacing system for cardiac protection is provided. One or more cardiac protection pacing sequences each including alternating pacing and non-pacing periods are timed. The pacing periods each have a pacing duration during which a plurality of pacing pulses is delivered from an implantable pulse generator. The non-pacing periods each having a non-pacing duration during which no pacing pulses is delivered from the implantable pulse generator. The pacing pulses are delivered from the implantable pulse generator to a coronary stent. The coronary stent includes at least one electrode portion electrically coupled to the implantable pulse generator. The electrode portion functions as a pacing electrode.

This Summary is an overview of some of the teachings of the present application and not intended to be an exclusive or exhaustive treatment of the present subject matter. Further details about the present subject matter are found in the detailed description and appended claims. Other aspects of the invention will be apparent to persons skilled in the art upon reading and understanding the following detailed description and viewing the drawings that form a part thereof. The scope of the present invention is defined by the appended claims and their legal equivalents.

### BRIEF DESCRIPTION OF THE DRAWINGS

The drawings illustrate generally, by way of example, various embodiments discussed in the present document. The drawings are for illustrative purposes only and may not be to scale.

FIG. 1 is an illustration of an embodiment of an implantable cardiac protection pacing system and portions of an environment in which the system is used.

FIG. 2 is an illustration of another embodiment of the implantable cardiac protection pacing system and portions of an environment in which the system is used.

FIG. 3 is an illustration of an embodiment of a pacing system including the implantable cardiac protection pacing system and an external system.

FIG. 4 is a block diagram illustrating an embodiment of portions of a circuit of the implantable system.

FIG. 5 is a block diagram illustrating a specific embodiment of portions of the circuit of the implantable system.

FIG. 6 is a block diagram illustrating another specific embodiment of portions of the circuit of the implantable system.

FIG. 7 is a block diagram illustrating an embodiment of portions of a circuit of the external system.

FIG. 8 is a flow chart illustrating an embodiment of a method for delivering pacing pulses for cardiac protection.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention. References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

This document discusses a pacing system that delivers pacing pulses to protect the heart from injuries associated with ischemic events, including MI. According to a cardiac protection pacing algorithm, pacing pulses are delivered to the heart to cause mechanical asynchrony in the myocardial contractions. The mechanical asynchrony increases the degree of cell stretch in the late contracting myocardial regions, thereby commencing an intracellular signaling cascade that temporarily protects the heart from an ischemic event. Many patients having suffered an MI or being at risk of an MI receive a vascular intervention treatment that leaves an intravascular device in a blood vessel where ischemia is likely to develop as the blood vessel becomes occluded. According to the present subject matter, a pacing system includes a pulse generator (PG) that is connected to an intravascular device to deliver pacing pulses by using at least a portion of the intravascular device as a pacing electrode. One example of the intravascular device is a coronary stent. The PG is incorporated into the coronary stent or is electrically connected to the coronary stent using a lead. The pacing system provides a means for cardiac protection pacing for a patient receiving the coronary stent. Such a means is particularly valuable when the patient neither has a pacemaker already implanted nor expects to have a pacemaker implanted for therapeutic purpose(s) other than the cardiac protection pacing. The cardiac protection pacing protects the patient's heart from tissue damage and development of heart failure associated with ischemic events, including MI. While the coronary stent is used as a specific example for discussion in this document, other intravascular devices suitable for conducting electrical pulses to the heart are each usable as one or more pacing electrodes according to the present subject matter.

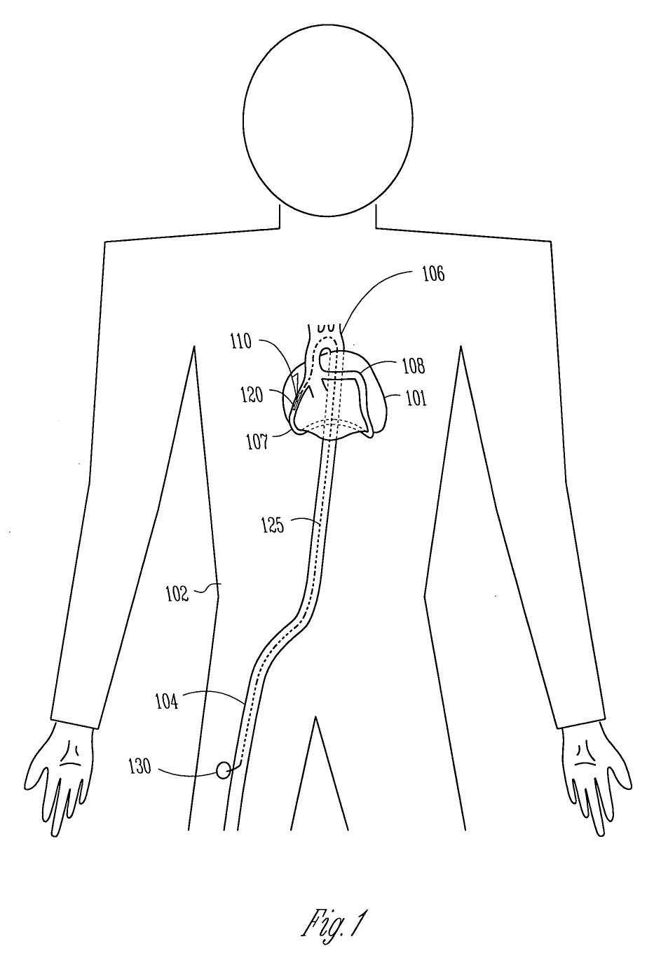

FIG. 1 is an illustration of an embodiment of an implantable system 110 and portions of an environment in which implantable system 110 is used. Implantable system 110 is an embodiment of an implantable cardiac protection pacing system that delivers cardiac protection pacing therapy to protect a heart 101 from injuries associated with ischemic events, including MI. In the illustrated embodiment, implantable system 110 includes a coronary stent 120 connected to an implantable PG 130 through a lead 125.

Coronary stent 120 is inserted into a coronary artery during a percutaneous transluminal coronary angioplasty (PTCA) procedure. During the PTCA procedure, an opening is made on a femoral artery 104 in a patient's body 102. An angioplasty device is inserted into femoral artery 104 and advanced to an aorta 106 and then to an occluded coronary artery to open up that coronary artery. Then, using a stent delivery catheter, coronary stent 120 is inserted into femoral artery 104 and advanced to aorta 106 and then to the coronary artery that has been opened up to be placed in that coronary artery. In the illustrated embodiment, coronary stent 120 is placed in a right coronary artery 107. In another embodiment, coronary stent 120 is placed in a left coronary artery 108.

Lead 125 is connected to coronary stent 120 before its insertion into femoral artery 104. As coronary stent 120 is placed the coronary artery, lead 125 is an intravascular lead extending from coronary stent 120 in the coronary artery through aorta 106 and femoral artery 104 to the opening on the femoral artery 104. After the placement of coronary stent 120 in the coronary artery, implantable PG 130 is subcutaneously implanted near the opening on the femoral artery 104. Lead 125 is then connected to implantable PG 130. By the end of the operation, implantable system 110 is completely implanted in body 102. In one embodiment, lead 125 has an elongate body having a length in a range of approximately 30 centimeters to 120 centimeters and a diameter in a range of approximately 0.125 millimeters to 1 millimeter. One or more insulated conductors extend through the elongate body to provide electrical connections between coronary stent 120 and implantable PG 130. To prevent blood coagulation, at least a portion of lead 125 is coated with an anti-coagulative agent.

Implantable PG 130 delivers pacing pulses by following a cardiac protection pacing sequence. The pacing pulses are delivered to heart 101 through lead 125 and coronary stent 120, which is used as a pacing electrode. The cardiac protection pacing sequence provides for cardiac protection pacing therapy before, during, and/or after an ischemic event to minimize cardiac injuries associated with the ischemic event.

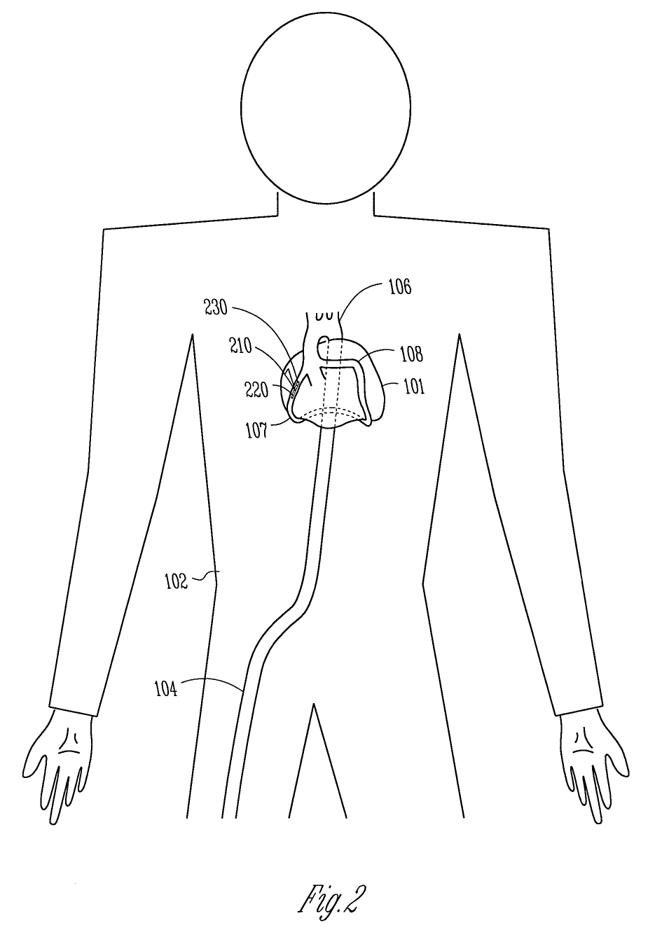

FIG. 2 is an illustration of an embodiment of an implantable system 210 and portions of an environment in which implantable system 210 is used. Implantable system 210 is another embodiment of the implantable cardiac protection pacing system that delivers cardiac protection pacing therapy to protect heart 101 from injuries associated with ischemic events, including MI. In the illustrated embodiment, implantable system 210 includes an implantable PG 230 attached to a coronary stent 220 to form an integrated intravascular PG-stent.

Implantable system 210 is inserted during a PTCA procedure. During the PTCA procedure, an opening is made on a femoral artery 104 in a patient's body 102. An angioplasty device is inserted into femoral artery 104 and advanced to an aorta 106 and then to an occluded coronary artery to open up that coronary artery. Then, using a stent delivery catheter, implantable system 210 is inserted into femoral artery 104 and advanced to aorta 106 and then to the coronary artery that has been opened up to be placed in that coronary artery. In the illustrated embodiment, implantable system 210 is placed in a right coronary artery 107. In another embodiment, implantable system 210 is placed in a left coronary artery 108.

Implantable PG 230 delivers pacing pulses by following the cardiac protection pacing sequence. The pacing pulses are delivered to heart 101 through coronary stent 220, which is used as a pacing electrode. The cardiac protection pacing sequence provides for cardiac protection pacing therapy before, during, and/or after an ischemic event to minimize cardiac injuries associated with the ischemic event.

Implantable PG 230 is sufficient small in size such that when implantable system 210 is placed in a coronary artery, the blood flow in that artery does not become a concern. In one embodiment, the size constraints requires that implantable PG 230 is externally powered using a telemetry link allowing for power transmission or includes a rechargeable battery that is rechargeable using the telemetry link, as further discussed below. In one embodiment, at least a portion of implantable PG 230 is coated with an anti-coagulative agent.

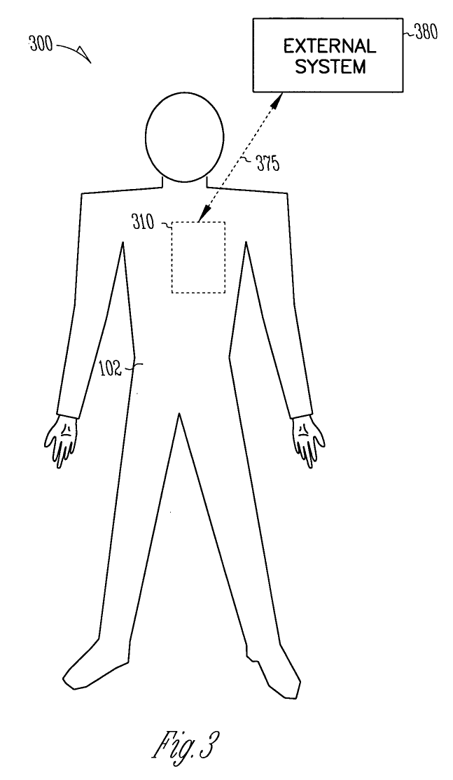

FIG. 3 is an illustration of an embodiment of a pacing system 300, which includes an implantable cardiac protection pacing system 310 and an external system 380. In various embodiments, implantable cardiac protection pacing system 310 includes one of implantable system 110 and implantable system 210. In various embodiments, in addition to functioning as a stent and delivering pacing pulses, implantable cardiac protection pacing system 310 also performs various physiological sensing and detection functions. A telemetry link 375 provides for wireless communication between implantable cardiac protection pacing system 310 and external system 380.

External system 380 allows for programming of implantable cardiac protection pacing system 310 and/or reception of signals acquired by implantable cardiac protection pacing system 310. In one embodiment, external system 380 includes a programmer. In another embodiment, external system 380 includes a hand-held controller. In another embodiment, external system 380 includes a patient management system. The patient monitoring system includes an external device communicating with implantable cardiac protection pacing system 310 via telemetry link 375, a telecommunication network coupled to the external device, and a remote device coupled to the telecommunication network. The remote device allows a user to control or program implantable cardiac protection pacing system 310 from a location remote from the patient.

Telemetry link 375 provides for data transmission from external system 380 to implantable cardiac protection pacing system 310. This may include, for example, programming implantable cardiac protection pacing system 310 to acquire physiological data, programming implantable cardiac protection pacing system 310 to deliver pacing pulses according to a predetermined pacing algorithm, and controlling delivery of pacing pulses using implantable cardiac protection pacing system 310. In various embodiments, telemetry link 375 also provides for data transmission from implantable cardiac protection pacing system 310 to external system 380. This may include, for example, transmitting real-time physiological data acquired by implantable cardiac protection pacing system 310, extracting physiological data acquired by and stored in implantable cardiac protection pacing system 310, extracting therapy history data stored in implantable cardiac protection pacing system 310, and extracting data indicating an operational status of implantable cardiac protection pacing system 310 (e.g., battery status). In one embodiment, in addition to data transmission, telemetry link 375 also provides for power transmission from external system 380 to implantable cardiac protection pacing system 310. The power transmission provides implantable cardiac protection pacing system 310 with the energy required for its operation. In one embodiment, telemetry link 375 is an inductive telemetry link. In an alternative embodiment, telemetry link 375 is a far-field radio-frequency (RF) telemetry link. In another alternative embodiment, telemetry link 375 is an ultrasonic telemetry link.

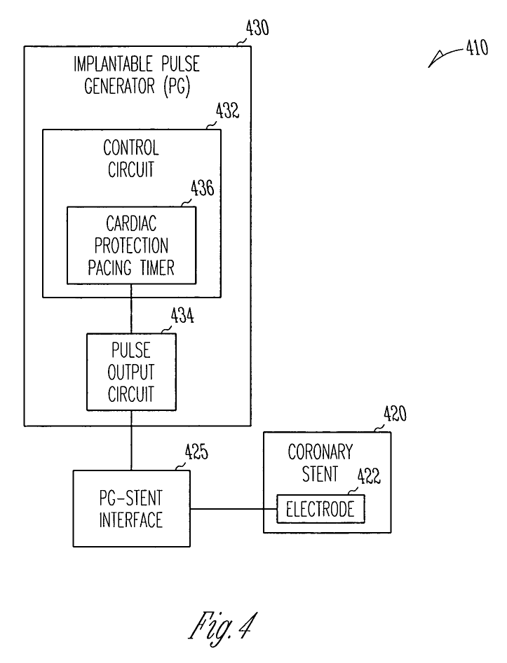

FIG. 4 is a block diagram illustrating an embodiment of portions of a circuit of an implantable system 410. Implantable system 410 is a specific embodiment of implantable cardiac protection pacing system 310 and includes an implantable PG 430, a PG-stent interface 425, and a coronary stent 420. In various embodiments, implantable system 110 and implantable system 210 each include the circuit illustrated in FIG. 4.

Implantable PG 430 is a specific embodiment of implantable PG 130 or 230 and includes electronic circuitry contained in a hermetically sealed implantable housing. Implantable PG 430 includes a control circuit 432 and a pulse output circuit 434. Control circuit 432 includes a cardiac protection pacing timer 436. Cardiac protection pacing timer 436 times a cardiac protection pacing sequence that controls the timing for delivering pacing pulses before, during, and/or after an ischemic event to minimize cardiac injuries associated with the ischemic event. In one embodiment, the cardiac protection pacing sequence includes alternating pacing and non-pacing periods. The pacing periods each have a pacing duration during which a plurality of pacing pulses is delivered in a predetermined pacing mode. The non-pacing periods each have a non-pacing duration during which no pacing pulse is delivered. In one embodiment, cardiac protection pacing timer 436 initiates and times cardiac protection pacing sequences according to a predetermined schedule, such as on a periodic basis. Pulse output circuit 434 delivers the plurality of pacing pulses during each of the pacing periods.

In one embodiment, cardiac protection pacing timer 436 times a postconditioning sequence after the ischemic event to minimize cardiac injuries associated with that ischemic event and a plurality of prophylactic preconditioning pacing sequences to minimize potential cardiac injuries associated with potentially recurrent ischemic events. The postconditioning sequence and the preconditioning sequence are each an instance of the cardiac protection pacing sequence timed by cardiac protection pacing timer 436. The postconditioning sequence includes alternating postconditioning pacing and non-pacing periods. The postconditioning pacing periods each have a postconditioning pacing duration during which a plurality of pacing pulses is delivered. The postconditioning non-pacing periods each have a postconditioning non-pacing duration during which no pacing pulse is delivered. The postconditioning sequence has a postconditioning sequence duration in a range of approximately 30 seconds to 1 hour, with approximately 10 minutes being a specific example. The postconditioning pacing duration is in a range of approximately 5 seconds to 10 minutes, with approximately 30 seconds being a specific example. The postconditioning non-pacing duration is in a range of approximately 5 seconds to 10 minutes, with approximately 30 seconds being a specific example. The prophylactic preconditioning pacing sequences each include alternating preconditioning pacing and non-pacing periods. The preconditioning pacing periods each have a preconditioning pacing duration during which a plurality of pacing pulse is delivered. The preconditioning non-pacing periods each have a preconditioning non-pacing duration during which no pacing pulse is delivered. The prophylactic preconditioning pacing sequences each have a preconditioning sequence duration in a range of approximately 10 minute to 1 hour, with approximately 40 minutes being a specific example. The preconditioning pacing duration is in a range of approximately 1 minute to 30 minutes, with approximately 5 minutes being a specific example. The preconditioning non-pacing duration is in a range of approximately 1 minute to 30 minutes, with approximately 5 minutes being a specific example. In one embodiment, the prophylactic preconditioning pacing sequences are initiated on a periodic basis, with a period in a range of approximately 30 minutes to 72 hours, with approximately 48 hours being a specific example. In one embodiment, cardiac protection pacing timer 436 includes a mode switch. When a cardiac protection pacing therapy is initiated in response to the ischemic event, cardiac protection pacing timer 436 is in a postconditioning timing mode during which the postconditioning sequence is timed. After the postconditioning sequence is completed, the mode switch switches the timing mode of cardiac protection pacing timer 436 from the postconditioning mode to a preconditioning mode during which the prophylactic preconditioning pacing sequences are timed.

Coronary stent 420 is a specific embodiment of coronary stent 120 or 220 and includes an electrode 422, which is electrically connected to pulse output circuit 434 through PG-stent interface 425 for the purpose of pacing pulse delivery. In one embodiment, coronary stent 420 has a conductive portion functioning as electrode 422. In other words, electrode 422 represents an electrode portion of coronary stent 420, i.e., the conductive portion that functions as a pacing electrode. In one embodiment, coronary stent 420 includes a bare metal frame. In another embodiment, coronary stent 420 includes a drug-coated metal frame. In another embodiment, coronary stent 420 includes portions made of bioreabsorbable material. In this embodiment, the implantable system configuration illustrated as implantable system 110 is more suitable than the implantable system configuration illustrated as implantable system 210. Implantable system 410 also includes a return electrode electrically connected to pulse output circuit 434 for the purpose of pacing pulse delivery. In one embodiment, a portion of the implantable housing that is electrically insulated from electrode 422 functions as the return electrode. In another embodiment, the return electrode is incorporated into coronary stent 420 and is electrically insulated from electrode 422.

PG-stent interface 425 electrically connects pulse output circuit 434 and electrode 422. In a specific embodiment, as illustrated in FIG. 1 (implantable system 110), PG-stent interface 425 includes a lead such as lead 125. The lead includes one or more insulated wires that electrically connect pulse output circuit 434 and electrode 422. Implantable PG 430 includes a connector on the implantable housing to provide for a detachable connection to the lead. This allows replacement of implantable PG 430, when needed, without the need to replace coronary stent 420 or PG-stent interface 425. In another specific embodiment, as illustrated in FIG. 2 (implantable system 210), PG-stent interface electrically connect pulse output circuit 434 and electrode 422 with the intravascular PG-stent. The implantable housing of implantable PG 430 is attached to coronary stent 420.

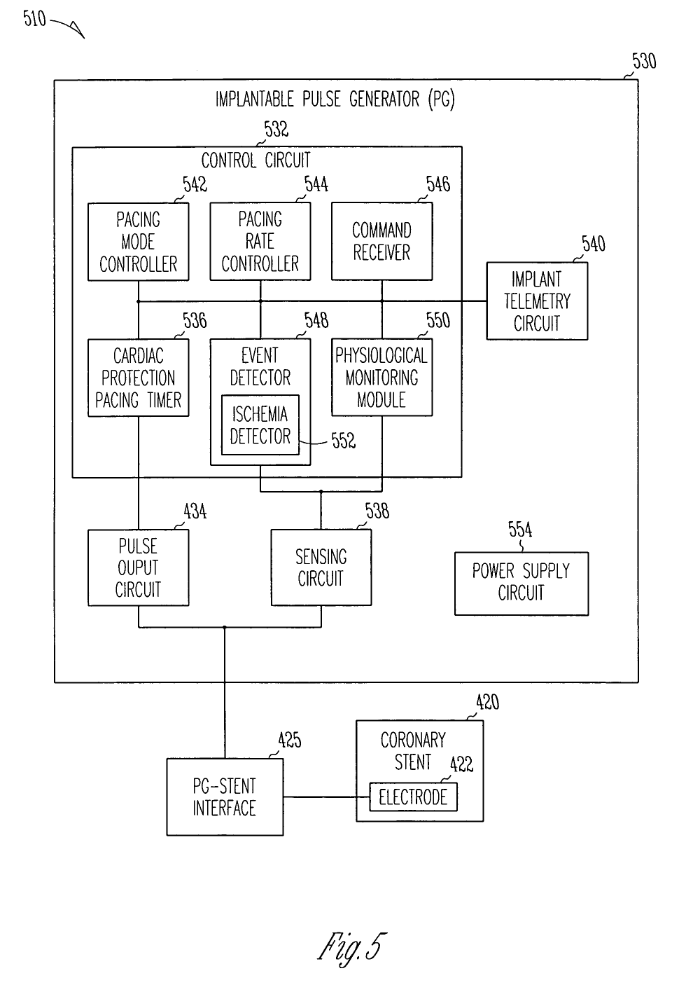

FIG. 5 is a block diagram illustrating an embodiment of portions of the circuit of an implantable system 510. Implantable system 510 is a specific embodiment of implantable system 410 and includes an implantable PG 530, PG-stent interface 425, and coronary stent 420. Implantable PG 530 is a specific embodiment of implantable PG 430 and includes a control circuit 532, pulse output circuit 434, a sensing circuit 538, an implant telemetry circuit 540, and a power supply circuit 554.

Control circuit 532 is a specific embodiment of control circuit 432 and includes cardiac protection pacing timer 536, a pacing mode controller 542, a pacing rate controller 544, a command receiver 546, an event detector 548, and a physiological monitoring module 550. In various embodiments, depending on the required or desirable functions of implantable system 510, control circuit 532 includes one or more of cardiac protection pacing time 536, pacing mode controller 542, pacing rate controller 544, command receiver 546, event detector 548, and physiological monitoring module 550. For example, if implantable system 510 is used to perform the limited function of delivering rapid pacing pulses in VOO mode at a fixed pacing rate for a fixed pacing period on a periodic basis with a fixed period, only cardiac protection pacing timer 536 is required.

Cardiac protection pacing timer 536 is a specific embodiment of cardiac protection pacing timer 436 and times the cardiac protection pacing sequence that controls the timing for delivering the pacing pulses before, during, and/or after an ischemic event to minimize cardiac injuries associated with the ischemic event. In one embodiment, the cardiac protection pacing sequence includes the alternating pacing and non-pacing periods. In one embodiment, cardiac protection pacing timer 536 initiates and times cardiac protection pacing sequences according to a predetermined schedule, such as on a periodic basis, as discussed above with respect to cardiac protection pacing timer 436. In another embodiment, cardiac protection pacing timer 536 initiates and times one or more cardiac protection pacing sequences in response to a pacing command received from command receiver 546. In one embodiment, the pacing command includes a single signal initiating a cardiac protection pacing sequence or a pacing period. In another embodiment, the pacing command includes a sequence of signals each initiating one of the pacing periods of the cardiac protection pacing sequence.

Pacing mode controller 542 controls the delivery of the pacing pulses during the pacing periods according to a predetermined pacing mode. In one embodiment, the pacing mode is programmable using external system 380. Examples of the pacing mode include the VOO and VVI pacing modes, including their rate adaptive versions if applicable. In various embodiments where cardiac sensing is required by the pacing mode, sensing circuit 538 senses an electrogram using electrode 422. In one embodiment, the pacing mode is a rate-adaptive pacing mode, and sensing circuit 538 senses an activity signal such as an acceleration signal using an accelerometer. In one embodiment, pacing mode controller 542 controls the delivery of the pacing pulses during the pacing periods in a ventricular rate regularization (VRR) pacing mode. The VRR mode refers to a pacing mode in which the delivery of pacing pulses is controlled according to a VRR algorithm. Examples of the VRR algorithm are discussed in U.S. patent application Ser. No. 09/316,515, entitled “METHOD AND APPARATUS FOR TREATING IRREGULAR VENTRICULAR CONTRACTIONS SUCH AS DURING ATRIAL ARRHYTHMIA,” filed on May 21, 1999 and U.S. Pat. No. 6,285,907, entitled “SYSTEM PROVIDING VENTRICULAR PACING AND BIVENTRICULAR COORDINATION,” both assigned to Cardiac Pacemakers, Inc., which are incorporated herein by reference in their entirety.

Pacing rate controller 544 controls the pacing rate during the pacing periods. In one embodiment, the pacing rate is in a range of approximately 50 pulses per minute (ppm) to 120 ppm. In a specific embodiment, the pacing rate is approximately 70 ppm. In one embodiment, pacing rate controller 544 sets the pacing rate higher than the intrinsic heart rate of the patient. In a specific embodiment, pacing rate controller 544 sets the pacing rate at approximate 20 ppm above the intrinsic heart rate of the patient. In one embodiment, pacing rate controller 544 dynamically adjusts the pacing rate in response to any substantial change in the intrinsic heart rate of the patient.

Pacing command receiver 546 receives the pacing command. In one embodiment, the pacing command is transmitted from external system 380, and pacing command receiver 546 receives the pacing command through implant telemetry circuit 540. In another embodiment, the pacing command is produced within implantable system 510 in response to a detected event that is predetermined to indicate a need for the cardiac protection pacing, and pacing command receiver 546 receives the pacing command from event detector 548. In response to the pacing command received by command receiver 546, cardiac protection pacing timer 536 initiates a pacing period or a cardiac protection pacing sequence. In one embodiment, the pacing command specifies the pacing duration, and cardiac protection pacing timer 536 times the pacing duration according to the pacing command.

Event detector 548 detects one or more predetermined type events indicative of a need for the cardiac protection pacing. In response to a detected predetermined type event, event detector 548 produces the pacing command. In one embodiment, event detector 548 includes an ischemia detector 552 that detects an ischemic event. In a specific embodiment, ischemia detector 552 detects the ischemic event from a cardiac signal sensed by sensing circuit 538. The cardiac signal is an electrogram sensed via electrode 422, through which the pacing pulses are also delivered. One example of an electrogram-based ischemia detector is discussed in U.S. patent application Ser. No. 09/962,852, entitled “EVOKED RESPONSE SENSING FOR ISCHEMIA DETECTION,” filed on Sep. 25, 2001, assigned to Cardiac Pacemakers, Inc., which is incorporated herein by reference in its entirety. In response to a detection of the ischemic event, event detector 548 produces the pacing command according to a predetermined timing relationship between the occurrence of an ischemic event and the delivery of the cardiac protection pacing. In one embodiment, event detector 548 issues the pacing command immediately in response to the detection of the ischemic event. In another embodiment, event detector 548 issues the pacing command after the end of the ischemic event as detected by ischemia detector 552. In response to the pacing command, cardiac protection pacing timer 536 initiates the pacing period or the cardiac protection pacing sequence.

Physiological signal monitoring module 550 monitors one or more physiological variables from one or more physiological signals sensed by sensing circuit 538. In one embodiment, sensing circuit 538 senses an elecotrogram using electrode 422. In a further embodiment, sensing circuit 538 senses additional one or more physiological signals using one or more sensors in, and/or connected to, implantable PG 530 and/or coronary stent 420. In one embodiment, the one or more physiological variables are transmitted to external system 380 through implant telemetry circuit 540. In another embodiment, event detector 548 detects the one or more predetermined type events based on the one or more physiological variables. In one embodiment, physiological signal monitoring module 550 includes a heart rate detector to detect a heart rate from the electrogram sensed by sensing circuit 538. In a further embodiment, physiological signal monitoring module 550 includes a heart rate variability (HRV) detector to detect HRV from the heart rate. The HRV detector produces an HRV parameter representative of the HRV based on the heart rate detected over a predetermined period of time.

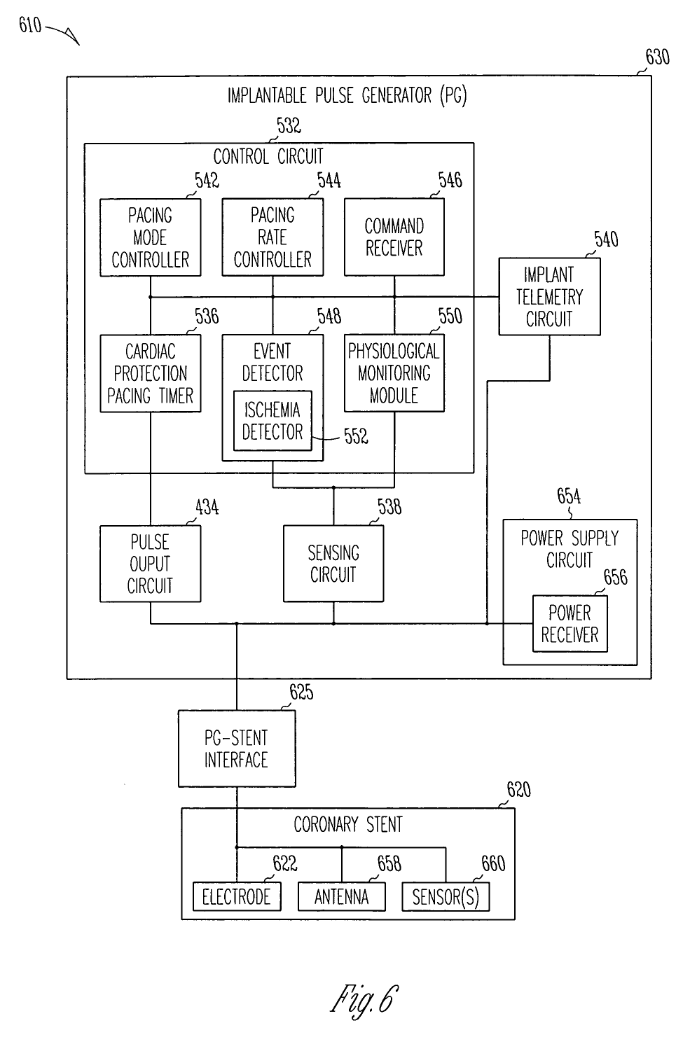

Power supply circuit 554 provides the circuitry of implantable PG 530 with the energy needed for its operation. In one embodiment, power supply circuit 554 includes a battery as the power source of implantable PG 530. In another embodiment, power supply circuit 554 receives power from external system 380, as discussed below with reference to FIG. 6. The choice of using a battery, receiving power from an external source, or both depends on factors including power consumption, size constraints, and intended longevity of implantable PG 530. In one embodiment, receiving power from an external source allows implantable PG 530 to be made small enough for use in an integrated intravascular PG-stent such as implantable system 210. In a specific embodiment, implantable PG 530 receives power from the external source and does not include a battery. In another embodiment, implantable PG 530 includes a small rechargeable battery and receives power from the external source to charge that rechargeable battery.

FIG. 6 is a block diagram illustrating an embodiment of portions of the circuit of an implantable system 610. Implantable system 610 is another specific embodiment of implantable system 410 and includes an implantable PG 630, a PG-stent interface 625, and a coronary stent 620. Implantable system 630 is powered by an external power source and includes substantially all the structural components of implantable system 530 to perform substantially all the functions of implantable system 530.

Power supply circuit 654 is a specific embodiment of power supply circuit 554 and includes a power receiver 656. Power receiver 656 receives RF power from an antenna 658, which receives RF power transmitted from external system 380 through telemetry link 375. Coronary stent 620 is a specific embodiment of coronary stent 420 and includes an electrode 622 and antenna 658. Electrode 622 represents an electrode portion of coronary stent 620, i.e., a conductive portion that functions as a pacing electrode. Antenna 658 represents an antenna portion of coronary stent 620, i.e., a conductive portion that functions as an antenna that receives RF power. In one embodiment, the electrode portion and the antenna portion include the same conductive portion of coronary stent 620. In other words, coronary stent 420 has a conductive portion functioning as electrode 622 and antenna 658. Power receiver 656 converts the received RF power to dc power to provide the circuitry of implantable system 610 with power for its operation. In a further embodiment, power supply circuit 654 includes a rechargeable battery and a battery charging circuit. When external system 380 is coupled to implantable system 610 via telemetry link 375, the battery charging circuit receives dc power from power receiver 656 and charges the rechargeable battery. When external system 380 is not coupled to implantable system 610 via telemetry link 375, the rechargeable battery provides the circuitry of implantable system 610 with power for its operation.

In one embodiment, antenna 658 is also used for data transmission using implantable telemetry circuit 540. In one embodiment, coronary stent 620 further includes one or more sensors 660 each used to sense a physiological signal to be received by sensing circuit 538 and/or physiological monitoring module 550. Examples of such sensor(s) include an activity sensor, a posture sensor, a respiratory rate sensor, a regional wall motion sensor, a stoke volume sensor, a pH sensor, a pressure sensor, an impedance sensor, and a strain sensor. In various embodiments, one or more physiological signals sensed by sensor(s) 660 are used for allowing an initiation of a cardiac protection pacing sequence. In a specific embodiment, the cardiac protection pacing sequence is initiated when such one or more physiological signals indicate that the patient is at rest. In another specific embodiment, the strain sensor is a strain gage sensor incorporated into coronary stent 620 to sense a signal indicative of bending forces applied onto the stent. The timing and amplitude of the bending forces reflects the cardiac wall motion in the region near the stent, and such regional cardiac wall motion indicates whether the region is ischemic. PG-stent interface 625 provides for all the connections required for transmitting RF power from antenna 658 to power receiver 656, transmitting data between antenna 658 and implant telemetry circuit 540, delivering the pacing pulses from pulse output circuit 434 to electrode 622, transmitting the electrogram from electrode 622 to sensing circuit 538, and transmitting other physiological signal(s), if any, from sensor(s) 660 to sensing circuit 538 and/or physiological monitoring module 550.

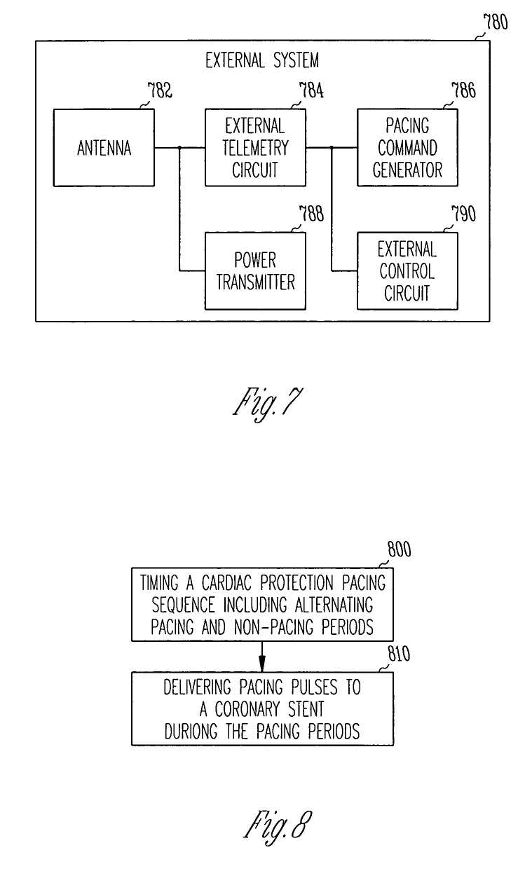

FIG. 7 is a block diagram illustrating an embodiment of portions of the circuit of an external system 780. External system 780 is a specific embodiment of external system 380 and includes an antenna 782, an external telemetry circuit 784, a pacing command generator 786, a power transmitter 788, and an external control circuit 790.

External telemetry circuit 784 transmits data to, and receives data from, implantable cardiac protection pacing system 310 (including its various embodiments) through antenna 782. Pacing command generator 786 generates the pacing command initiating the pacing period(s) or the cardiac protection pacing sequence. The pacing command is transmitted to implantable cardiac protection pacing system 310 through external telemetry circuit 784 and antenna 782. In one embodiment, external system 780 includes a user interface to receive user commands, and pacing command generator 786 produces the pacing command according to one or more user commands. External control circuit 790 controls the operation of external system 780. In one embodiment, external control circuit 790 receives data indicative of a need to initiate the pacing period(s) or the cardiac protection pacing sequence from implantable cardiac protection pacing system 310. The data represent, for example, an event detected by event detector 548 or a physiological variable produced by physiological monitoring module 550. In response, external control circuit 790 causes pacing command generator 786 to generate the pacing command. In one embodiment in which implantable cardiac protection pacing system 310 is powered by an external power source, power transmitter 788 generates RF power (an RF signal carrying the power needed to operate the implantable system) and transmits the RF power through antenna 782. In one embodiment, the data transmission using telemetry link 375 is performed by modulating the RF signal carrying the power. In one embodiment, power transmitter 788 generates and transmits the RF power in a form of magnetic energy. In another embodiment, power transmitter 788 generates and transmits the RF power in a form of electromagnetic energy. In one embodiment, power transmitter 788 generates and transmits the RF power in a form of acoustic (ultrasonic) energy.

FIG. 8 is a flow chart illustrating an embodiment of a method for delivering pacing pulses for cardiac protection before, during, and/or after an ischemic event, including MI. In one embodiment, the method is performed using implantable cardiac protection pacing system 310, including its various embodiments.

A cardiac protection pacing sequence is timed at 800. The cardiac protection pacing sequence includes alternating pacing and non-pacing periods. The pacing periods each have a pacing duration during which a plurality of pacing pulses is delivered in a predetermined pacing mode. The non-pacing periods each have a non-pacing duration during which no pacing pulse is delivered. Examples of the pacing modes include the VOO, VVI, and VRR pacing modes. In one embodiment, the pacing rate is set higher than the patient's intrinsic heart rate. In one embodiment, the pacing rate is dynamically adjusted in response to any substantial change in the patient's intrinsic heart rate, such as in the VRR mode. In one embodiment, the pacing periods are initiated according to a predetermined schedule, such as on a periodic basis according to a predetermined period. In another embodiment, a pacing command is received. The cardiac protection pacing sequence, and/or each of the pacing periods of the cardiac protection pacing sequence, is initiated in response to the pacing command. In a further embodiment, the pacing duration is also set according to the pacing command. In one embodiment, the pacing command is issued by a user. In another embodiment, a predetermined type event indicative of a need for the cardiac protection pacing is detected. In response to the detection of such a predetermined type event, the pacing command is produced. In a specific embodiment, the predetermined type event includes an ischemic event.

The plurality of pacing pulses in each of the pacing periods is delivered from an implantable PG to a coronary stent at 810. The coronary stent includes an electrode portion functioning as a pacing electrode. In one embodiment, the pacing pulses are delivered to that electrode portion of the coronary stent through a lead providing electrical connection between the coronary stent and the implantable PG. In one embodiment, the power required to operate the implantable PG is provided by a battery within the implantable PG. In another embodiment, the power required to operate the implantable PG is received from an external power source in the form of magnetic, electromagnetic, or acoustic energy.

In various embodiments, steps 800 and 810 are repeated after an ischemic event. A postconditioning sequence is timed after the ischemic event to minimize cardiac injuries associated with that ischemic event. Then, a plurality of prophylactic preconditioning pacing sequences is timed to minimize potential cardiac injuries associated with potentially recurrent ischemic events. The postconditioning sequence and the preconditioning sequence are each an instance of the cardiac protection pacing sequence. The postconditioning sequence includes alternating postconditioning pacing and non-pacing periods. The postconditioning pacing periods each have a postconditioning pacing duration during which a plurality of pacing pulses is delivered. The postconditioning non-pacing periods each have a postconditioning non-pacing duration during which no pacing pulse is delivered. The prophylactic preconditioning pacing sequences each include alternating preconditioning pacing and non-pacing periods. The preconditioning pacing periods each have a preconditioning pacing duration during which a plurality of pacing pulse is delivered. The preconditioning non-pacing periods each have a preconditioning non-pacing duration during which no pacing pulse is delivered.

It is to be understood that the above detailed description is intended to be illustrative, and not restrictive. Other embodiments will be apparent to those of skill in the art upon reading and understanding the above description. The scope of the invention should, therefore, be determined with reference to the appended claims, along with the full scope of equivalents to which such claims are entitled.

What is claimed is:

1. A cardiac pacing system, comprising:
an implantable pulse generator including:
a control circuit including a cardiac protection pacing timer adapted to time one or more cardiac protection pacing sequences each including alternating pacing and non-pacing periods, the pacing periods each having a pacing duration during which a plurality of pacing pulses is delivered, the non-pacing periods each having a non-pacing duration during which no pacing pulse is delivered; and
a pulse output circuit, coupled to the control circuit, to deliver the plurality of pacing pulses during each of the pacing periods; and
a coronary stent including at least one electrode portion electrically connected to the pulse output circuit for delivering the plurality of pacing pulses during the each of the pacing periods.

2. The system of claim 1, wherein the implantable pulse generator is attached to the coronary stent to form an integrated intravascular pulse generator-stent.

3. The system of claim 1, wherein the implantable pulse generator is configured for subcutaneous placement, and further comprising a lead providing for the electrical connection between the at least one electrode portion of the coronary stent and the pulse output circuit of the implantable pulse generator.

4. The system of claim 3, wherein the lead comprises an intravascular lead having a length in a range of approximately 30 centimeters to 120 centimeters and a diameter in a range of approximately 0.125 millimeters to 1 millimeter and including at least a portion coated with an anti-coagulative agent.

5. The system of claim 1, wherein the control circuit comprises a pacing mode controller adapted to control the delivery of the plurality of pacing pulses during the each of the pacing periods in a VOO mode.

6. The system of claim 1, further comprising a sensing circuit, coupled to the at least one electrode portion of the coronary stent, to sense an electrogram, and wherein the control circuit comprises a pacing mode controller adapted to control the delivery of the plurality of pacing pulses during the each of the pacing periods in a VVI mode.

7. The system of claim 1, further comprising a sensing circuit, coupled to the at least one electrode portion of the coronary stent, to sense an electrogram, and wherein the control circuit comprises a pacing mode controller adapted to control the delivery of the plurality of pacing pulses during the each of the pacing periods in a ventricular rate regularization (VRR) mode.

8. The system of claim 1, wherein the cardiac protection pacing timer is adapted to initiate the one or more cardiac protection pacing sequences according to a predetermined schedule.

9. The system of claim 1, wherein the cardiac protection pacing timer is adapted to time a postconditioning sequence of the one or more cardiac protection pacing sequences during a postconditioning timing mode, switch the postconditioning timing mode to a preconditioning timing mode, and time a plurality of prophylactic preconditioning pacing sequences of the one or more cardiac protection pacing sequences during the preconditioning timing mode.

10. The system of claim 1, wherein the control circuit comprises a command receiver to receive a pacing command, and the cardiac protection pacing timer is adapted to initiate at least one of the one or more cardiac protection pacing sequences in response to the pacing command.

11. The system of claim 10, wherein the control circuit comprises an event detector to detect a predetermined type event and produce the pacing command in response to the detection of the predetermined type event.

12. The system of claim 11, wherein the event detector comprises an ischemia detector adapted to detect an ischemic event.

13. The system of claim 10, wherein the implantable pulse generator comprises an implant telemetry circuit, coupled to the command receiver, to receive the pacing command.

14. The system of claim 1, further comprising an external system communicatively coupled to the implantable pulse generator, the external system including a power transmitter adapted to transmit radio frequency (RF) power to the implantable pulse generator, and wherein the implantable pulse generator comprises:
an antenna configured to receive the RF power, the antenna including at least an antenna portion of the coronary stent; and
a power supply circuit, coupled to the antenna, to convert the RF power to a dc power.

15. The system of claim 14, wherein the power supply circuit comprises:
a rechargeable battery; and
a battery charging circuit, coupled to the rechargeable battery, to charge the rechargeable battery using the dc power.

16. The system of claim 1, further comprising a strain sensor incorporated into the coronary stent and coupled to the control circuit, the strain sensor adapted to sense a signal indicative of bending forces applied onto the coronary stent.

17. A method for operating a pacing system, comprising:
timing one or more cardiac protection pacing sequences each including alternating pacing and non-pacing periods, the pacing periods each having a pacing duration during which a plurality of pacing pulses is delivered from an implantable pulse generator, the non-pacing periods each having a non-pacing duration during which no pacing pulses is delivered from the implantable pulse generator; and
delivering the plurality of pacing pulses to a coronary stent during each of the pacing periods, the coronary stent including at least one electrode portion electrically coupled to the implantable pulse generator and functioning as a pacing electrode.

18. The method of claim 17, wherein delivering the plurality of pacing pulses comprises delivering the plurality of pacing pulses to the coronary stent through an intravascular lead.

19. The method of claim 17, further comprising delivering the plurality of pacing pulses in a VOO mode.

20. The method of claim 17, further comprising:
sensing a cardiac signal using the electrode portion of the coronary stent; and
delivering the plurality of pacing pulses in a VVI mode.

21. The method of claim 17, further comprising:
sensing a cardiac signal using the electrode portion of the coronary stent; and
delivering the plurality of pacing pulses in a ventricular rate regularization (VRR) mode.

22. The method of claim 17, wherein delivering the plurality of pacing pulses comprises setting a pacing rate to approximately 20 pulses per minute higher than an intrinsic heart rate.

23. The method of claim 17, wherein timing the one or more cardiac protection pacing sequences comprises timing a postconditioning sequence of the one or more cardiac protection pacing sequences, the postconditioning sequence having a postconditioning sequence duration in a range of approximately 30 seconds to 1 hour and including alternating postconditioning pacing and non-pacing periods, the postconditioning pacing periods each having a postconditioning pacing duration in a range of approximately 5 seconds to 10 minutes during which the plurality of pacing pulses is delivered, the postconditioning non-pacing periods each having a postconditioning non-pacing duration in a range of approximately 5 seconds to 10 minutes during which no pacing pulse is delivered.

24. The method of claim 17, wherein timing the one or more cardiac protection pacing sequences comprises timing a plurality of prophylactic preconditioning pacing sequences of the one or more cardiac protection pacing sequences, the prophylactic preconditioning pacing sequences each having a preconditioning sequence duration in a range of approximately 10 minutes to 1 hour and including alternating preconditioning pacing and non-pacing periods, the preconditioning pacing periods each having a preconditioning pacing duration in a range of approximately 1 minute to 30 minutes during which the plurality of pacing pulses is delivered, the preconditioning non-pacing periods each having a preconditioning non-pacing duration in a range of approximately 1 minute to 30 minutes during which no pacing pulse is delivered.

25. The method of claim 24, wherein timing the plurality of prophylactic preconditioning pacing sequences comprises initiating the prophylactic preconditioning pacing sequences on a periodic basis using a predetermined period in a range of approximately 30 minutes to 72 hours.

26. The method of claim 17, wherein timing the one or more cardiac protection pacing sequences comprises timing a postconditioning sequence of the one or more cardiac protection pacing sequences during a postconditioning timing mode, switching the postconditioning timing mode to a preconditioning timing mode, and timing a plurality of prophylactic preconditioning pacing sequences of the one or more cardiac protection pacing sequences during the preconditioning timing mode.

27. The method of claim 17, wherein timing the one or more cardiac protection pacing sequences comprises:
receiving a pacing command; and
initiating at least one of the one or more cardiac protection pacing sequences in response to the pacing command.

28. The method of claim 27, further comprising:
detecting a predetermined type event; and
producing the pacing command in response to the detection of the predetermined type event.

29. The method of claim 28, wherein detecting the predetermined type event comprises detecting an ischemic event.

30. The method of claim 27, further comprising receiving the pacing command from a user.

31. The method of claim 17, further comprising receiving radio frequency (RF) power for operating the implantable pulse generator, and wherein the coronary stent includes an antenna portion used as an antenna for receiving the RF power.

32. The method of claim 31, further comprising charging a rechargeable battery using the received RF power.
